(19) United States
(12) Patent Application Publication
(10) Pub. No.: US 2019/0321324 A1
(43) Pub. Date: Oct. 24, 2019

(54) QUATERNARY AMINE ANTIBIOTIC THERAPEUTICS

(71) Applicant: BOARD OF REGENTS, THE UNIVERSITY OF TEXAS SYSTEM, Austin, TX (US)

(72) Inventors: Bryan W. DAVIES, Austin, TX (US); Gregory A. KNAUF, Lindsay, TX (US)

(21) Appl. No.: 16/474,603

(22) PCT Filed: Dec. 29, 2017
(86) PCT No.: PCT/US2017/068909

§ 371 Date: Jun. 28, 2019

Related U.S. Application Data
(60) Provisional application No. 62/440,953, filed on Dec. 30, 2016.

(51) Int. Cl. A61K 31/245; A61K 9/00; A61K 45/06

(52) U.S. Cl. CPC A61K 31/245; A61K 45/06; A61K 9/0019; A61K 9/0053

(57) ABSTRACT

In one aspect, the present disclosure provides methods of treating a bacterial infection using a salt of the formula (I) wherein X₁ is a monovalent anion. These compounds may be used to treat infections of bacteria including those which are resistant to one or more commonly used antibiotics such as vancomycin or methicillin.

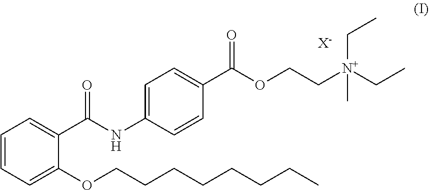

This application claims the benefit of priority to U.S. Provisional Application Ser. No. 62/440,953, filed on Dec. 30, 2016, the entire contents of which are incorporated herein by reference.

### BACKGROUND

### 1. Field

This disclosure relates to the fields of medicine, pharmacology, and antimicrobial activity. In particular, methods of treatment of a bacterial infection.

### 2. Related Art

Infectious diseases are currently the second leading cause of death worldwide and the third leading cause of death in economically advanced countries despite the development of antibiotics (Nathan, 2004). Furthermore, the threatening emergence of bacterial resistance to many antibiotics presents a serious challenge for their clinical use. As our antibiotic options continue to shrink, prevention becomes increasing important in elevating the burden of hospital-associated bacterial infections (Otter et al., 2011; Weber et al., 2010; Barsanti and Woeltje, 2009). Surface decontamination relies heavily on the use of biocides; chemical agents with antimicrobial properties. Among the biocide classes, quaternary amine compounds (QACs) such as benzalkonium chloride (BAC), are frequently used for disinfecting medical equipment, hospital surfaces, patient wounds, and healthcare workers' hands. QACs are recommended for use at concentrations many times their minimal inhibitory concentration (MIC) and effectively eradicate vegetative bacteria at these doses under laboratory conditions. But practical clinical conditions including high bacterial density, the presence of biofilm and organic matter, and high ion concentrations (Klimek and Bailey, 1955; Otter et al., 2015) dramatically decrease the effectiveness of QAC biocides, which allows bacteria to escape death and persist in the hospital environment. Despite their wide use, the antimicrobial effects of QACs are unclear, especially at near inhibitory concentrations. Additionally, the incomplete understanding of QAC action has fueled concerns of cross-resistance between biocides and antibiotics. If this were to occur, resistant bacteria could be selected both during antibiotic treatment and clinical disinfection, promoting the propagation of resistant endemic strains.

Therefore, there remains a need to develop new methods of treating bacterial infections especially methods which involve different mechanisms.

### SUMMARY

Thus, the present disclosure provides methods of treating a bacteria infection comprising administering to the patient a therapeutically effective amount of otilonium salt.

In some aspects, the present disclosure provides methods of treating a bacterial infection in a patient comprising administering to the patient in need thereof a therapeutically effective amount of a compound of the formula:

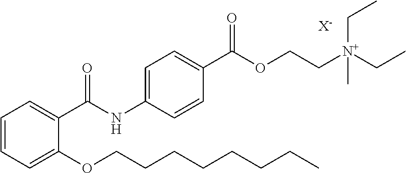

wherein:

X is a monovalent anion.

In some embodiments, the monovalent anion is a halide such as bromide or chloride. The compound may be further defined as:

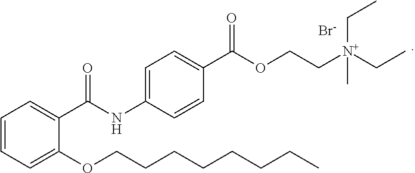 [SMILES: CCCCCCCCOc1ccccc1C(=O)Nc1ccc(C(=O)OCC[N+](C)(CC)CC)cc1 ; [Br-]]

The infection may be a hospital acquired infection. In other embodiments, the infection may be of a gram negative bacteria such as a pathogenic gram negative bacteria. The gram negative bacteria may be Pseudomonas aeruginosa, Acinetobacter baumannii, Stenotrophomonas maltophilia, Escherichia coli, or Legionella pneumophila. In other embodiments, the infection is of a gram positive bacteria. The gram positive bacteria may be a pathogenic gram positive bacteria such as Clostridium difficile, Enterococcus faecalis, Enterococcus faecium, Staphylococcus aureus, or Staphylococcus epidermidis. In other embodiments, the infection is of a gram indeterminate bacteria. The gram indeterminate bacteria may be a pathogenic gram indeterminate bacteria such as Mycobacterium tuberculosis. In other embodiments, the gram indeterminate bacteria causes tuberculosis.

The bacteria may be resistant to one or more antibiotics such as vancomycin, a β-lactam antibiotic, or a carbapenem. In some embodiments, the β-lactam antibiotic is methicillin. In some embodiments, the compound is administered orally. In other embodiments, the compound is administered via injection.

In still yet another aspect, the present disclosure provides methods of inhibiting the growth of a bacterium comprising contacting the bacterium with an effective amount of a compound of the formula:

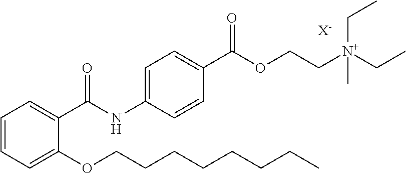

wherein:

X is a monovalent anion.

In some embodiments, the monovalent anion is a halide such as bromide or chloride. The compound may be further defined as:

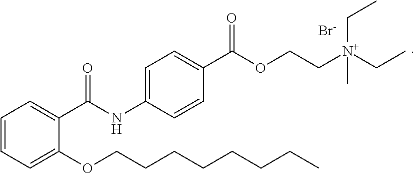 [SMILES: CCCCCCCCOc1ccccc1C(=O)Nc1ccc(C(=O)OCC[N+](C)(CC)CC)cc1 ; [Br-]]

In yet another aspect, the present disclosure provides methods of killing a bacterium comprising contacting the bacterium with an effective amount of a compound of the formula:

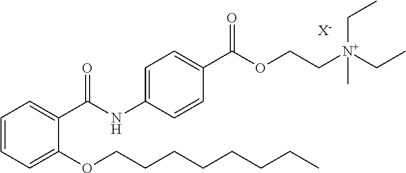

wherein:

X is a monovalent anion.

In some embodiments, the monovalent anion is a halide such as bromide or chloride. The compound may be further defined as:

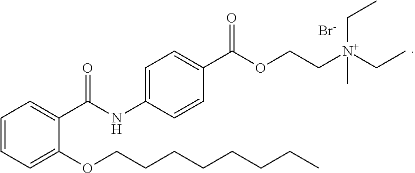 [SMILES: CCCCCCCCOc1ccccc1C(=O)Nc1ccc(C(=O)OCC[N+](C)(CC)CC)cc1 ; [Br-]]

The methods may be performed in vitro, performed ex vivo, or performed in vivo. In some embodiments, the methods are sufficient to treat a disease or disorder. The methods may further comprise a second antibiotic therapy. In some embodiments, the methods comprise administering the compound once. In other embodiments, the methods comprise administering the compound two or more times. The patient may be a mammal such as a human.

In still yet another aspect, the present disclosure provides pharmaceutical compositions comprising:

(A) a compound of the formula:

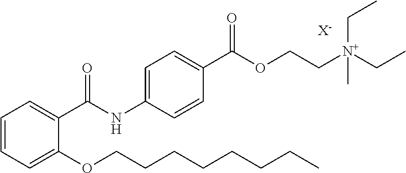

wherein:
X is a monovalent anion;
   (B) an excipient;
   wherein the pharmaceutical composition is formulated for administration by injection or oral administration. In some embodiments, the compound is further defined as:

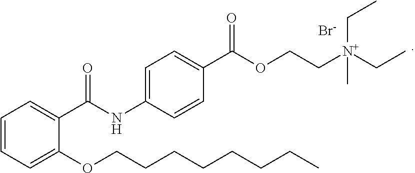 [SMILES: CCCCCCCCOc1ccccc1C(=O)Nc1ccc(C(=O)OCC[N+](C)(CC)CC)cc1 ; [Br-]]

The pharmaceutical composition may be formulated for administration by intraarterial injection, intraperitoneal injection, intravenous injection, or subcutaneous injection. In some embodiments, the excipient is a pharmaceutically acceptable carrier such as a saline solution. In other embodiments, the pharmaceutical composition is formulated for oral administration.

In some embodiments, the pharmaceutical composition is formulated as a unit dose. The unit dose may be from about 0.1 μg/mL to about 100 μg/mL or from about 1 μg/mL to about 50 μg/mL. The unit dose may be about 0.5 mg to about 150 mg, about 10 mg to about 90 mg, or about 20 mg to about 80 mg. In some embodiments, the unit dose is about 40 mg, 50 mg, 60 mg, 70 mg, or 80 mg.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description and examples provided herewith.

FIG. 1 shows the chemical structures of bretylium, clofilium, and otilonium.

FIG. 2 shows Otilonium Bromide (OB) drives lethal proteome damage. Protein aggregates from A. baumannii bacteria grown with or without Otilonium Bromide treatment. Aggregates were extracted from an equal number of A. baumannii cells under each treatment condition. The experiment was repeated at least three times with a representative result shown.

FIG. 3 shows the effects of various concentrations of vancomycin and Otilonium bromide (OB) on two different strains of C. difficile, Strain 43244 and Strain 630 (MDR). These experiments were carried out in triplicate for each therapeutic agent and strain.

FIG. 4 confirms the production of both toxin A and toxin B in the C. difficile strains used in the studies carried herein.

### DESCRIPTION OF ILLUSTRATIVE EMBODIMENTS

In some aspects, the present disclosure provides methods of using a quaternary amine compound, an otilonium salt. In particular, the present disclosure relates to using otilonium bromide to treat a bacterial infection. The bacteria infection may be an infection of a gram positive, gram negative, or gram indeterminate bacteria. These bacteria may be resistant to one or more commonly used antibiotic such as vancomycin, methicillin, or a β-lactamase.

### I. Bacterial Infections

In some aspects of the present disclosure, the compounds disclosed herein may be used to treat a bacterial infection. While humans contain numerous different bacteria on and inside their bodies, an imbalance in bacterial levels or the introduction of pathogenic bacteria can cause a symptomatic bacterial infection. Pathogenic bacteria cause a variety of different diseases including but not limited to numerous foodborne illness, typhoid fever, tuberculosis, pneumonia, syphilis, and leprosy.

Additionally, different bacteria have a wide range of interactions with body and those interactions can modulate ability of the bacteria to cause an infection. For example, bacteria can be conditionally pathogenic such that they only cause an infection under specific conditions. For example, Staphylococcus and Streptococcus bacteria exist in the normal human bacterial biome, but these bacteria when they are allowed to colonize other parts of the body causing a skin infection, pneumonia, or sepsis. Other bacteria are known as opportunistic pathogens and only cause diseases in a patient with a weakened immune system or another disease or disorder.

Bacteria can also be intracellular pathogens which can grow and reproduce within the cells of the host organism. Such bacteria can be divided into two major categories as either obligate intracellular parasites or facultative intracellular parasites. Obligate intracellular parasites require the host cell in order to reproduce and include such bacteria as but are not limited to Chlamydophila, Rickettsia, and Ehrlichia which are known to cause pneumonia, urinary tract infections, typhus, and Rocky Mountain spotted fever. Facultative intracellular parasites can reproduce either intracellular or extracellular. Some non-limiting examples of facultative intracellular parasites include Salmonella, Listeria, Legionella, Mycobacterium, and Brucella which are known to cause food poisoning, typhoid fever, sepsis, meningitis, Legionnaire's disease, tuberculosis, leprosy, and brucellosis.

The compounds described herein may be used in the treatment of bacterial infections, including those caused by Staphylococcus aureus. S. aureus is a major human pathogen, causing a wide variety of illnesses ranging from mild skin and soft tissue infections and food poisoning to life-threatening illnesses such as deep post-surgical infections, septicaemia, endocarditis, necrotizing pneumonia, and toxic shock syndrome. These organisms have a remarkable ability to accumulate additional antibiotic resistance determinants, resulting in the formation of multiply-drug-resistant strains.

Methicillin, being the first semi-synthetic penicillin to be developed, was introduced in 1959 to overcome the problem of penicillin-resistant S. aureus due to β-lactamase (penicillinase) production (Livermore, 2000). However, methicillin-resistant S. aureus (MRSA) strains were identified soon after the introduction of methicillin (Barber, 1961; Jevons, 1961). The methods described herein may be used in the treatment of MRSA bacterial strains.

Additionally, the compounds of the present disclosure may be used to treat a Streptococcus pneumoniae infection. Streptococcus pneumoniae is a gram-positive, alpha-hemolytic, bile soluble aerotolerant anaerobe and a member of the genus Streptococcus. A significant human pathogenic bacterium, S. pneumoniae was recognized as a major cause of pneumonia in the late 19th century and is the subject of many humoral immunity studies.

Despite the name, the organism causes many types of pneumococcal infection other than pneumonia, including acute sinusitis, otitis media, meningitis, bacteremia, sepsis, osteomyelitis, septic arthritis, endocarditis, peritonitis, pericarditis, cellulitis, and brain abscess. S. pneumoniae is the most common cause of bacterial meningitis in adults and children, and is one of the top two isolates found in ear infection, otitis media. Pneumococcal pneumonia is more common in the very young and the very old.

S. pneumoniae can be differentiated from S. viridans, some of which are also alpha hemolytic, using an optochin test, as S. pneumoniae is optochin sensitive. S. pneumoniae can also be distinguished based on its sensitivity to lysis by bile. The encapsulated, gram-positive coccoid bacteria have a distinctive morphology on gram stain, the so-called, “lancet shape.” It has a polysaccharide capsule that acts as a virulence factor for the organism; more than 90 different serotypes are known, and these types differ in virulence, prevalence, and extent of drug resistance.

S. pneumoniae is part of the normal upper respiratory tract flora but as with many natural flora, it can become pathogenic under the right conditions (e.g., if the immune system of the host is suppressed). Invasins such as Pneumolysin, an anti-phagocytic capsule, various adhesins and immunogenic cell wall components are all major virulence factors.

Finally, bacterial infections could be targeted to a specific location in or on the body. For example, bacteria could be harmless if only exposed to the specific organs, but when it comes in contact with a specific organ or tissue, the bacteria can begin replicating and cause a bacterial infection.

A. Gram-Positive Bacteria

In some aspects of the present disclosure, the compounds disclosed herein may be used to treat a bacterial infection by a gram-positive bacteria. Gram-positive bacteria contain a thick peptidoglycan layer within the cell wall which prevents the bacteria from releasing the stain when dyed with crystal violet. Without being bound by theory, the gram-positive bacteria are often more susceptible to antibiotics. Generally, gram-positive bacteria, in addition to the thick peptidoglycan layer, also comprise a lipid monolayer and contain teichoic acids which react with lipids to form lipoteichoic acids that can act as a chelating agent. Additionally, in gram-positive bacteria, the peptidoglycan layer is outer surface of the bacteria. Many gram-positive bacteria have been known to cause disease including, but are not limited to, Streptococcus, Straphylococcus, Corynebacterium, Enterococcus, Listeria, Bacillus, Clostridium, Rathybacter, Leifsonia, and Clavibacter.

B. Gram-Negative Bacteria

In some aspects of the present disclosure, the compounds disclosed herein may be used to treat a bacterial infection by a gram-negative bacteria. Gram-negative bacteria do not retain the crystal violet stain after washing with alcohol. Gram-negative bacteria, on the other hand, have a thin peptidoglycan layer with an outer membrane of lipopolysaccharides and phospholipids as well as a space between the peptidoglycan and the outer cell membrane called the periplasmic space. Gram-negative bacterial generally do not have teichoic acids or lipoteichoic acids in their outer coating. Generally, gram-negative bacteria also release some endotoxin and contain prions which act as molecular transport units for specific compounds. Most bacteria are gram-negative. Some non-limiting examples of gram-negative bacteria include Bordetella, Borrelia, Burcelia, Campylobacteria, Escherichia, Francisella, Haemophilus, Helicobacter, Legionella, Leptospira, Neisseria, Pseudomonas, Rickettsia, Salmonella, Shigella, Treponema, Vibrio, and Yersinia.

C. Gram-Indeterminate Bacteria

In some aspects of the present disclosure, the compounds disclosed herein may be used to treat a bacterial infection by a gram-indeterminate bacteria. Gram-indeterminate bacteria do not full stain or partially stain when exposed to crystal violet. Without being bound by theory, a gram-indeterminate bacteria may exhibit some of the properties of the gram-positive and gram-negative bacteria. A non-limiting example of a gram-indeterminate bacteria include Mycobacterium tuberculosis or Mycobacterium leprae.

### II. Therapies

A. Pharmaceutical Formulations and Routes of Administration Where clinical applications are contemplated, it will be necessary to prepare pharmaceutical compositions in a form appropriate for the intended application. In some embodiments, such a formulation with the otilonium salt is contemplated. Generally, this will entail preparing compositions that are essentially free of pyrogens, as well as other impurities that could be harmful to humans or animals.

One will generally desire to employ appropriate salts and buffers to render the salts stable and allow for uptake by target cells. Buffers also will be employed when recombinant cells are introduced into a patient. Aqueous compositions of the present disclosure comprise an effective amount of the salt to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active salts may also be administered parenterally or intraperitoneally. Solutions of the active compounds as a pharmacologically acceptable salts can be prepared in water and may be optionally mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration, the otilonium salts of the present disclosure may be incorporated with excipients or used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 mL of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA's Division of Biological Standards and Quality Control of the Office of Compliance and Biologics Quality.

B. Methods of Treatment

In particular, the compositions that may be used in treating microbial infections in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing the death of bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection and other drugs being administered concurrently. A composition as described herein is typically administered at a dosage that inhibits the growth or proliferation of a bacterial cell or inhibits the growth of a biofilm as assayed by identifying a reduction in hematological parameters (complete blood count—CBC). In some embodiments, amounts of the salts used to inhibit bacterial growth is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the salts may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

C. Combination Therapies

It is envisioned that the otilonium salts described herein may be used in combination therapies with an additional antimicrobial agent such as an antibiotic or a compound which mitigates one or more of the side effects experienced by the patient.

These therapies would be provided in a combined amount effective to achieve a reduction in one or more disease parameter(s). This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the otilonium salts described herein may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Agents or factors suitable for use in a combined therapy with agents according to the present disclosure against an infectious disease include antibiotics such as penicillins, cephalosporins, carbapenems, macrolides, aminoglycosides, quinolones (including fluoroquinolones), sulfonamides and tetracylcines. Other combinations are contemplated.

1. Antibiotics

The term “antibiotics” are drugs which may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

The first commercially available antibiotic was released in the 1930's. Since then, many different antibiotics have been developed and widely prescribed. In 2010, on average, 4 in 5 Americans are prescribed antibiotics annually. Given the prevalence of antibiotics, bacteria have started to develop resistance to specific antibiotics and antibiotic mechanisms. Without being bound by theory, the use of antibiotics in combination with another antibiotic may modulate resistance and enhance the efficacy of one or both agents.

In some embodiments, antibiotics can fall into a wide range of classes. In some embodiments, the compounds of the present disclosure may be used in conjunction with another antibiotic. In some embodiments, the compounds may be used in conjunction with a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

### III. Definitions

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedrally substituted carbon atoms), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its (R) form, (S) form, or as a mixture of the (R) and (S) form, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

### IV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Bacterial Inhibition

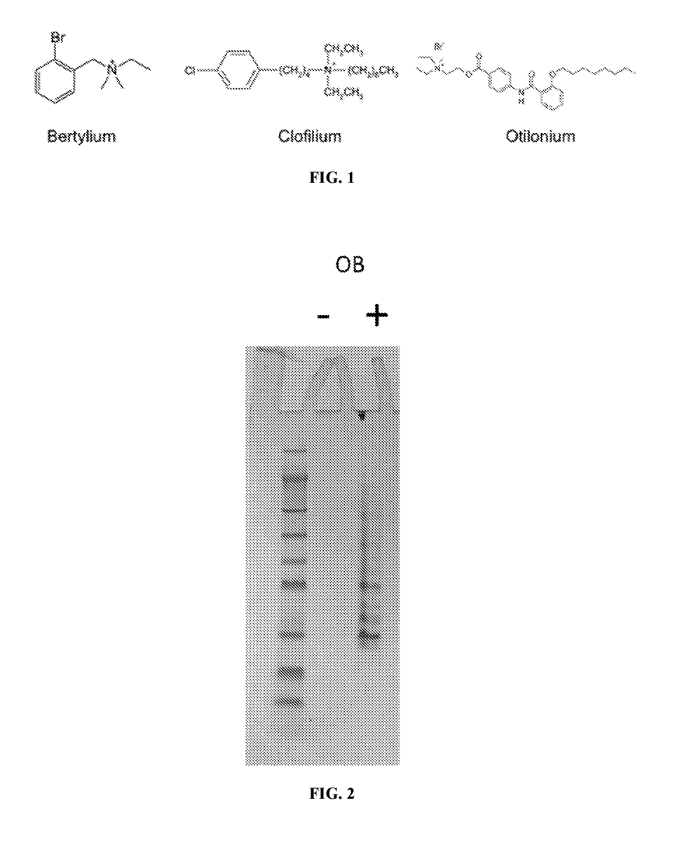

The antimicrobial activity of three QACs that have been approved for human use; Bretylium tosylate, Clofilium tosylate, and Otilonium bromide, were tested (FIG. 1). The length of the hydrocarbon tail on BAC is known to strongly influence its behavior. Clofilium tosylate and Otilonium bromide both have hydrocarbon tails that are hypothesized to allow cell envelope penetration. Indeed, both Clofilium tosylate and Otilonium bromide were found to prevent growth of both antibiotic resistant and susceptible A. baumannii, while Bretylium Tosylate, which lacks a hydrocarbon tail, did not show activity (Table 1). In fact, the MIC for Otilonium bromide was equivalent to that of BAC for strain 17978. Similar activity was determine for these three compounds in S. aureus (Table 2)

TABLE 1
A. baumannii MIC of clinically used QACs.

| | | | | Mutant | Mutant 7 | Mutant 1 | Mutant |
|---|---|---|---|---|---|---|---|
| | Strain | 17978 | AYE (MDR) | 8 (3) | (19) | (20) | 4 (23) |
| Compound | | | | | | | |
| Bertylium tosylate | | >256 | >256 | >256 | >256 | >256 | >256 |
| Clofilium tosylate | | 128 | 128 | 256 | 256 | 128 | 128 |
| Otilonium bromide | | 16 | 16 | 16 | 16 | 16 | 16 |

TABLE 2
S. aureus MIC of clinically used QACs.

| | Strain | Newman | MU50 (MDR) |
|---|---|---|---|
| Compound | | | |
| Bertylium tosylate | | >256 | >256 |
| Clofilium tosylate | | 16 | 32 |
| Otilonium bromide | | 2 | 4 |

BAC, Bretylium tosylate, Clofilium tosylate, and Otilonium bromide are all QACs but with different structures. This suggests that these compounds could interact with the ribosome in slightly different ways. Bretylium tosylate, Clofilium tosylate, and Otilonium bromide were tested on BAC resistant mutants representing mutations in ribosomal proteins S11, L23, L24 and the UTR of the ribosomal operon. Mutants with changes to S11 and L24 (Mutants 1 and 4 respectively) showed increased resistance to Clofilium tosylate indicating an overlap with BAC in ribosomal interaction. The MIC to Otilonium bromide was not affected in these mutants. The structure of Otilonium bromide is quite different from BAC suggesting it could interact with different areas. However, Otilonium bromide was found to cause a similar increase in A. baumannii aggregates, and that the lon mutant was highly sensitized to Otilonium bromide treatment, suggesting a similar global effect on the ribosome and proteostais (FIG. 2).

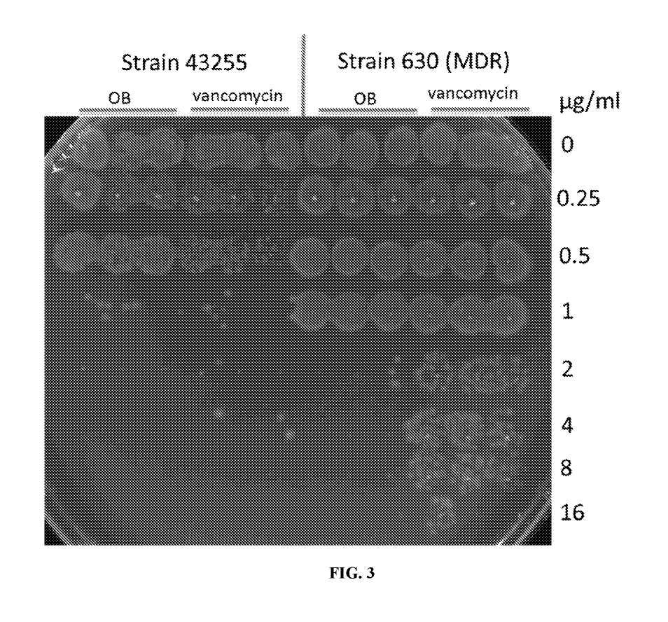

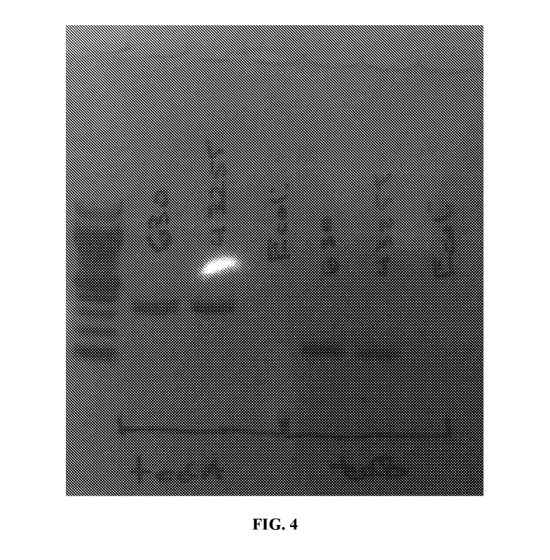

Activity of Otilonium bromide was also tested using two strains of C. difficile, 43255 and 630 (MDR). The minimal bactericidal assay was performed using 5×10⁵ C. difficile bacteria where incubated with vancomycin or OB at the indicated concentrations for 20 h at 37° C. in an anaerobic chamber. The total volume per well in BHIS medium was 100 μl and 5 μL was spotted on RCM medium and plates were incubated for 24 h. The MBC is identified as the concentration of compound the reduced cfu to single or no numbers. Biological triplicate results are shown in FIG. 3 and Table 3. The C. difficile strains were tested to confirm that the strains produce both C. difficile toxin A and toxin B. FIG. 4 shows that both strains of C. difficile used in these studies produce both toxin A and B.

TABLE 3
C. difficile MBC of Otilonium bromide and Vancomycin

| | Strains | 43255 (μg/mL) | 630 (μg/mL) |
|---|---|---|---|
| Compounds | | | |
| Otilonium bromide | | 1 | 2 |
| Vancomycin | | 1 | ~2 |

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. References

The following references, to the extent that they provide exemplary procedural or other details supplementary to those set forth herein, are specifically incorporated herein by reference:
Anderson, N. G., Practical Process Research & Development—A Guide For Organic Chemists, 2^{nd} ed., Academic Press, New York, 2012.
Antibiotic Resistance Threats in the United States, 2013,
Austin-Ward and Villaseca, Rev. Med. Chil., 126(7):838-45, 1998.
Barber, J. Clin. Pathol., 14:385-393, 1961.
Barsanti and Woeltje, Infect. Dis. Clin. N. Am., 23:703-725, 2009.
Bukowski et al., Clin. Cancer Res., 4(10):2337-47, 1998.
Christodoulides et al., Microbiology, 144(Pt 11):3027-37, 1998.
Davidson et al., J. Immunother., 21(5):389-98, 1998.
Greene's Protective Groups in Organic Chemistry, Wuts and Greene, Ed., 1973
Hanibuchi et al., Int. J. Cancer, 78(4):480-485, 1998.
Hellstrand et al., Acta Oncol., 37(4):347-353, 1998.
Hui and Hashimoto, Infect. Immun., 66(11):5329-36, 1998.
Jevons, British Med J., 1:124-125, 1961.
Ju et al., Gene Ther., 7(19):1672-1679, 2000.
Klimek and Bailey, Applied Microbiology, 4(1):53-59, 1956
Livermore, Int. J. Antimicrob. Agents, 16(1:)S3-10, 2000.
March's Advanced Organic Chemistry: Reactions, Mechanisms, and Structure, 2007.
Mitchell et al., Ann. NY Acad Sci., 690:153-166, 1993.
Mitchell et al. J Clin. Oncol., 8(5):856-869, 1990.
Morton et al., Arch. Surg., 127:392-399, 1992.
Nathan, Nature, 431:899-902, 2004.
Otter, et al., J. Hospital Infection, 89:16-27, 2015.
Otter, et al., Infection Control and Hospital Epidemiology, 32(7):687-699, 2011.
Pangborn et al., Organometallics, 15:1518-1520, 1996.
Pietras et al., Oncogene, 17(17):2235-49, 1998.
Qin et al., Proc. Natl. Acad. Sci. USA, 95(24):14411-14416, 1998.
Ravindranath and Morton, Intern. Rev. Immunol., 7: 303-329, 1991.
Remington's Pharmaceutical Sciences, 15^{th} Ed., 1035-1038 and 1570-1580, 1990.
Remington's Pharmaceutical Sciences, 15^{th} Ed., 3:624-652, 1990.
Rosenberg et al., Ann. Surg. 210(4):474-548, 1989.
Rosenberg et al., N. Engl. J. Med, 319:1676, 1988.
Still et al., J. Org. Chem., 43:2923-2925, 1978.
U.S. Pat. No. 5,739,169
U.S. Pat. No. 5,801,005
U.S. Pat. No. 5,824,311
U.S. Pat. No. 5,830,880
U.S. Pat. No. 5,846,945
US Department of Health and Human Services, Center of Disease Control and Prevention, www.cdc.gov/drugresistance/threat report-2013/pdf/ar-threats-2013-508.pdf
Weber, et al., American Journal of Infection Control, 38:S25-S33, 2010

What is claimed is:

1. A method of treating a bacterial infection in a patient comprising administering to the patient in need thereof a therapeutically effective amount of a compound of the formula: 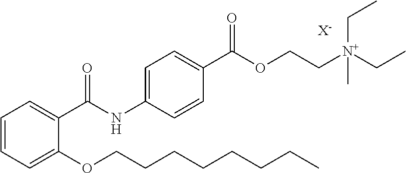
wherein:
X is a monovalent anion.

2. The method of claim 1, wherein the monovalent anion is a halide.

3. The method of claim 2, wherein the halide is a bromide or chloride.

4. The method according to any one of claims 1-3, wherein the compound is further defined as:

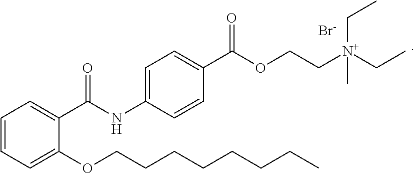 [SMILES: CCCCCCCCOc1ccccc1C(=O)Nc1ccc(C(=O)OCC[N+](C)(CC)CC)cc1 ; [Br-]]

5. The method according to any one of claims 1-4, wherein the infection is a hospital acquired infection.

6. The method according to any one of claims 1-5, wherein the infection is of a gram negative bacteria.

7. The method of claim 6, wherein the gram negative bacteria is a pathogenic gram negative bacteria.

8. The method of claim 7, wherein the gram negative bacteria is Pseudomonas aeruginosa, Acinetobacter baumannii, Stenotrophomonas maltophilia, Escherichia coli, or Legionella pneumophila.

9. The method according to any one of claims 1-5, wherein the infection is of a gram positive bacteria.

10. The method of claim 9, wherein the gram positive bacteria is a pathogenic gram positive bacteria.

11. The method of claim 10, wherein the gram positive bacteria is Clostridium difficile, Enterococcus faecalis, Enterococcus faecium, Staphylococcus aureus, or Staphylococcus epidermidis.

12. The method according to any one of claims 1-4, wherein the infection is of a gram indeterminate bacteria.

13. The method of claim 12, wherein the gram indeterminate bacteria is a pathogenic gram indeterminate bacteria.

14. The method of claim 13, wherein the gram indeterminate bacteria is Mycobacterium tuberculosis.

15. The method of claim 14, wherein the gram indeterminate bacteria causes tuberculosis.

16. The method according to any one of claims 1-15, wherein the bacteria is resistant to one or more antibiotics.

17. The method of claim 16, wherein the bacteria is resistant to vancomycin, a β-lactam antibiotic, or a carbapenem.

18. The method of claim 17, wherein the β-lactam antibiotic is methicillin.

19. The method according to any one of claims 1-18, wherein the compound is administered orally.

20. The method according to any one of claims 1-18, wherein the compound is administered via injection.

21. A method of inhibiting the growth of a bacterium comprising contacting the bacterium with an effective amount of a compound of the formula: 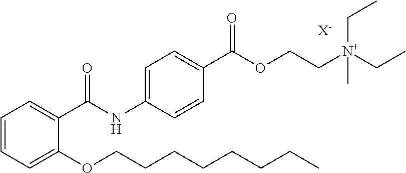
wherein:
X is a monovalent anion.

22. The method of claim 21, wherein the monovalent anion is a halide.

23. The method of claim 22, wherein the halide is a bromide or chloride.

24. The method according to any one of claims 21-23, wherein the compound is further defined as:

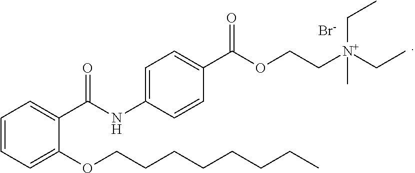 [SMILES: CCCCCCCCOc1ccccc1C(=O)Nc1ccc(C(=O)OCC[N+](C)(CC)CC)cc1 ; [Br-]]

25. A method of killing a bacterium comprising contacting the bacterium with an effective amount of a compound of the formula: 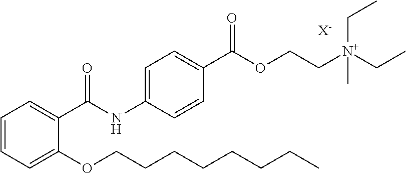
wherein:
X is a monovalent anion.

26. The method of claim 25, wherein the monovalent anion is a halide.

27. The method of claim 26, wherein the halide is a bromide or chloride.

28. The method according to any one of claims 25-27, wherein the compound is further defined as:

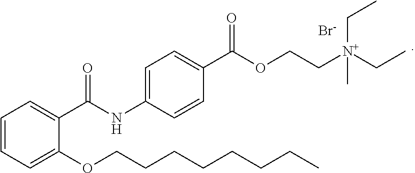 [SMILES: CCCCCCCCOc1ccccc1C(=O)Nc1ccc(C(=O)OCC[N+](C)(CC)CC)cc1 ; [Br-]]

29. The method according to any one of claims 21-28, wherein the method is performed in vitro.

30. The method according to any one of claims 21-28, wherein the method is performed ex vivo.

31. The method according to any one of claims 21-28, wherein the method is performed in vivo.

32. The method according to any one of claims 21-28, 30, and 31, wherein the method is sufficient to treat a disease or disorder.

33. The method according to any one of claims 1-32, wherein the method further comprises a second antibiotic therapy.

34. The method according to any one of claims 1-33, wherein the method comprises administering the compound once.

35. The method according to any one of claims 1-33, wherein the method comprises administering the compound two or more times.

36. The method according to any one of claims 1-20, wherein the patient is a mammal.

37. The method of claim 36, wherein the patient is a human.

38. A pharmaceutical composition comprising:
(A) a compound of the formula: 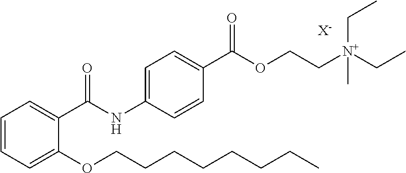
wherein:
X is a monovalent anion;
(B) an excipient;
wherein the pharmaceutical composition is formulated for administration by injection or oral administration.

39. The pharmaceutical composition of claim 38, wherein the compound is further defined as:

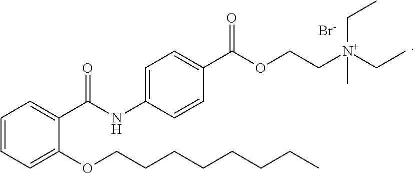 [SMILES: CCCCCCCCOc1ccccc1C(=O)Nc1ccc(C(=O)OCC[N+](C)(CC)CC)cc1 ; [Br-]]

40. The pharmaceutical composition of either claim 38 or claim 39, wherein the pharmaceutical composition is formulated for administration by intraarterial injection, intraperitoneal injection, intravenous injection, or subcutaneous injection.

41. The pharmaceutical composition according to any one of claims 38-40, wherein the excipient is a pharmaceutically acceptable carrier.

42. The pharmaceutical composition of claim 41, wherein the pharmaceutically acceptable carrier is a saline solution.

43. The pharmaceutical composition of claim 38, wherein the pharmaceutical composition is formulated for oral administration.

44. The pharmaceutical composition according to any one of claims 38-42, wherein the pharmaceutical composition is formulated as a unit dose.

45. The pharmaceutical composition of claim 44, wherein the unit dose is from about 0.1 ng/mL to about 100 μg/mL.

46. The pharmaceutical composition of claim 45, wherein the unit dose is from about 1 μg/mL to about 50 μg/mL.

47. The pharmaceutical composition of claim 44, wherein the unit dose is about 0.5 mg to about 150 mg.

48. The pharmaceutical composition of claim 47, wherein the unit dose is about 10 mg to about 90 mg.

49. The pharmaceutical composition of claim 48, wherein the unit dose is about 20 mg to about 80 mg.

50. The pharmaceutical composition according to any one of claims 38-49, wherein the pharmaceutical composition is formulated for treatment of a bacterial infection.
